(19) United States
(12) Patent Application Publication
(10) Pub. No.: US 2026/0275430 A1
(43) Pub. Date: Sep. 17, 2026

(54) METHOD FOR DETERMINING THE PRESENCE OR ABSENCE OF THE 4 DENGUE VIRUS SEROTYPES IN A BIOLOGICAL SAMPLE AND FOR DISTINGUISHING TWO DENGUE 2 VIRUS STRAINS, USING TWO PAIRS OF PRIMERS AND SNPS, AND, KIT FOR DETECTING DENGUE VIRUS SEROTYPES AND FOR DISTINGUISHING TWO DENV-2 STRAINS

(71) Applicant: FUNDAÇÃO OSWALDO CRUZ, RIO DE JANEIRO (BR)

(72) Inventors: PATRICIA CARVALHO DE SEQUEIRA SEQUEIRA, RIO DE JANEIRO (BR); ANNE ALINE PEREIRA DE PAIVA, RIO DE JANEIRO (BR); ANA MARIA BISPO DE FILIPPIS, RIO DE JANEIRO (BR); ZILTON FARIAS MEIRA VASCONCELOS, RIO DE JANEIRO (BR); NIELI RODRIGUES DA COSTA FARIA, ITAGUAI (BR); Simone Alves SAMPAIO, RIO DE JANEIRO (BR); LETICIA DA CUNHA GUIDA, RIO DE JANEIRO (BR); LEONARDO HENRIQUE FERREIRA GOMES, NITERÓI (BR)

(21) Appl. No.: 19/469,016

(22) PCT Filed: Mar. 27, 2024
(86) PCT No.: PCT/BR2024/050126

§ 371 Date: Sep. 25, 2025

(30) Foreign Application Priority Data
Mar. 29, 2023 (BR) 1020230059201

(51) Int. Cl. C12Q 1/70; C12Q 1/48; C12Q 1/6827; C12Q 1/686; C12Q 1/6876

(52) U.S. Cl. CPC C12Q 1/701; C12Q 1/48; C12Q 1/6827; C12Q 1/686; C12Q 1/6876; C12Y 207/07; C12Q 2600/156; C12Q 2600/16

(57) ABSTRACT

The present invention relates to a method for determining the presence or absence of the four serotypes of the dengue virus and differentiating between lineages I and II of serotype 2 of the dengue virus in a biological sample. The present invention also relates to a method for detecting the four serotypes of DENV. The present invention also relates to the use of two pairs of primers or SNPs to determine the presence or absence of the four serotypes of DENV or to differentiate between the lineages of serotype 2 of the dengue virus in a biological sample. The present invention also relates to a kit for detecting the four serotypes of the dengue virus or for distinguishing two strains of DENV-2.

### FIELD OF INVENTION

The present invention is in the field of diagnostic medicine and epidemiology. The present invention relates to a method for determining the presence or absence of the four serotypes of the dengue virus (DENV-1, DENV-2, DENV-3, and DENV-4) and, at the same time, identifying the variants (lineage I and lineage II) of the dengue virus 2 (DENV-2) present in a biological sample, using the known SNP sequences of each variant. The present invention also relates to a kit for detecting the different dengue virus serotypes and for distinguishing two DENV-2 virus lineages.

### BACKGROUND OF THE INVENTION

1. DENV: Etiological agent

DENV is an arbovirus belonging to the Flaviviridae family and the Flavivirus genus (Nedjadi T et al., 2015). Like other Flaviviruses, it has a genome consisting of single-stranded RNA (ssRNA) with positive polarity (Kuh R J et al, 2002). It contains only a single open reading frame (ORF) and is approximately 11 kb in length (Chambers T J et al, 1990). The viral particle is spherical, approximately 50 nm in diameter, and has a lipid envelope (Lupi O et al, 2007). Its genomic RNA is translated into a single polyprotein precursor, consisting of three structural proteins (C [capsid], prM [membrane], and E [envelope]) and seven non-s (NS1, NS2a, NS2b, NS3, NS4a, NS4b, and NS5) (Assenberg R et al, 2009).

### 1.2 Genetic Diversity of DENV

Based on phylogenetic analyses, four antigenically distinct serotypes have been determined for DENV: DENV-1, DENV-2, DENV-3, and DENV-4 (Vasilakis N &amp; Weaver S C, 2008). Each of these serotypes has distinct genotypes, resulting from the variability presented. Five genotypes are described for DENV-1 (Rico-Hesse R 1990; Weaver S C & Vasilakis N 2009; Chen R & Vasilakis N 2011), six genotypes for DENV-2 (Weaver S C & Vasilakis N 2009; Chen R & Vasilakis N 2011; RicoHesseR 1997), five genotypes for DENV-3 (Lanciotti R et al, 1994), and four genotypes for DENV-4 (Chen R & Vasilakis N 2011; Lanciotti R S et al 1997; Villabona-Arenas C J et al 2011).

### 1.3 DENV Replication Cycle

The viral replication process of members of the Flavivirus genus involves a large arsenal of mechanisms to be effective and consists of triggering a series of events at the permissive host cell level, which are: adsorption, penetration, denudation, translation, replication, assembly, and budding of viral particles (Selisko B et al 2014). Permissive host cells include monocytes, macrophages, and dendritic cells, and adsorption begins after the viral particle binds to the cell surface via the E protein (Sampath A & Padmanabhan R 2009). Virions are internalized by clathrin-mediated endocytosis (penetration), and the process of denudation-s begins through fusion between the viral and cell membranes (Araújo JMG et al 2015). Factors such as low endosomal pH and the participation of glycoproteins are essential for the disassembly of the particle and the release of genetic material. Subsequently, the viral messenger RNA is translated into a single polyprotein, which will give rise to structural and non-structural proteins. The transcription stage of viral genomic RNA ensures the continuity of the cycle, originating an intermediate RNA strand with negative polarity, in order to provide the template for the process to proceed (Lindenbach B D et al 2001). The replication process occurs in the endoplasmic reticulum, in the so-called “cytoplasmic viral factories,” and the virions are released by budding, undergoing maturation during transport through the vesicles of the Golgi apparatus. Parallel to the glycosylation of the E protein, prM undergoes cleavage by a furin, due to the low pH of the trans-Golgi network (TGN), becoming the M protein. Finally, the virions are released into the cytoplasm by exocytosis (Selisko B et al 2014).

### 1.4 Clinical Manifestations of DENV Infection

Infections caused by the dengue virus vary from individual to individual and may be asymptomatic or symptomatic. The clinical course of symptomatic disease can present as dengue with warning signs or severe dengue. Dengue with warning signs is the main clinical presentation involving fever and body aches and is self-limiting. Severe dengue can present with severe plasma leakage, severe hemorrhage, shock, and organ compromise (Tewari V V et al, 2018).

### 1.5 Molecular Epidemiology of DENV Viruses

According to the Rico-Hesse classification (2003), the four serotypes of the dengue virus have the following genotypes: DENV-1: Thailand, Asia, South Pacific, Americas/Africa, and Malaysia (Wild); DENV2: Malaysia/Subcontinental India, Southeast Asia, Americas, and West Africa; DENV-3: Southeast Asia/South Pacific, Thailand, Subcontinental India, and Americas; DENV-4: Indonesia, Southeast Asia, and Malaysia.

### 1.5 Molecular Epidemiology of DENV-2

Specifically in relation to DENV-2, molecular epidemiological analyses of this serotype report that the genotype circulating in Brazil is Asian/American. DENV-2 was introduced in Brazil in 1990 and reemerged in 2007, causing a large-scale epidemic. This reemergence of DENV-2 in 2007 caused a notable change in the clinical pattern of DENV infections in Brazil, when an increase in morbidity and mortality rates was observed. Molecular epidemiology studies showed that the reemergent DENV-2 was a viral variant that, despite belonging to the same genotype as the 1990 strain, was grouped in a monophyletic group different from the variants known until then.

Thus, two lineages within the Asian/American genotype have been identified and described to date: lineage I, from the viral variants that had been circulating in Brazil since 1990, and lineage II, from the reemergence period (Oliveira M F et al., 2010; Drumond B P 2013).

### 2. Laboratory Diagnosis of DENV

Currently, although a large proportion of diagnoses for DENV infections are often made solely on the basis of clinical management (Chan H &How C 2017), the main methods for laboratory identification of infections with signs and symptoms compatible with DENV infections revolve around serological and molecular assays (Kuno G et al., 1987, Lanciotti et al., 1992).

The use of serological methods, based on the detection of IgM and IgG antibodies produced after the first five days of illness, represents the most common methodology for the diagnosis of Flavivirus. However, this methodology has proven to be highly challenging for the diagnosis of DENV, especially in regions with an epidemiological scenario such as Brazil, where different arboviruses co-circulate. We frequently observe a high rate of cross-antigenic reactivity between different Flavivirus, which can provide false-positive results (Fatima Z et al 2011). In parallel with IgM and IgG testing, detection of the NS1 antigen by ELISA is also an effective approach for the early diagnosis of DENV (Banerjee A et al 2018).

Molecular methods for detecting the viral genome are highly sensitive and specific, as they are able to identify the presence of the viral genome in the early stages of the disease, with no cross-reactivity observed between arboviruses. Thus, the main method for diagnosing different DENV serotypes is based on reverse transcription followed by polymerase chain reaction (RT-PCR) (Lanciotti et al., 1992). More recently, conventional RT-PCR technology has evolved, and currently, the real-time RT-PCR technique based on TaqMan®-type fluorescent probes is the “flagship” of DENV molecular diagnosis (Araújo JMG et al., 2015).

Because the DENV genome is a positive-strand RNA, molecular diagnostic techniques use the action of the reverse transcriptase enzyme to construct complementary DNA (cDNA) strands from viral RNA and subsequently detect the viral genome (Lanciotti et al., 1992, 2007). This enzymatic reaction is standardized in RT-PCR assays that use RNA as the genome and has been used for DENV amplification since 1992 (Lanciotti et al., 1992). However, the present invention was able to identify DENV-2 variants without the use of reverse transcriptase, which is an advantage in terms of cost and detection time.

### 2.1 Real-Time PCR Followed by High-Resolution Melting Curve Analysis (PCR-HRM)

Conventionally, the genomic sequencing method is the standard tool used for DENV characterization and genotypic surveillance studies. However, this methodology is costly and requires a lot of time and staff training to accurately identify circulating genotypes and viral lineages. With the advancement of molecular PCR methodologies for viral genome detection, PCR-HRM (high resolution melting) is emerging as a sensitive, automated, and rapid alternative for detecting and differentiating viral variants. This method uses the PCR technique, but is capable of detecting small nucleotide variations (polymorphisms) (SNPs-Single-nucleotide polymorphism) through the different dissociation temperatures generated by these variations. In this way, different variants can be detected by a PCR-based method that does not require genomic sequencing. (Cam B V et al., 2001; Endy T P et al., 2002; Staikowsky F et al., 2008).

In view of this, PCR-HRM aims to strengthen the virological surveillance systems of arbovirus diagnostic laboratories in the country, contributing to the monitoring of the emergence and reemergence of DENV-2 lineages (Bharaj P et al., 2006).

Dissociation curve analysis is performed after PCR completion, without the need to open the reaction tube, and is characterized by the analysis of the transition behavior from double-stranded to single-stranded (dissociation) of the generated amplicons. The gradual increase in temperature at the end of PCR results in the separation of DNA strands and, consequently, the disassociation of double-stranded DNA dye molecules, causing a reduction in fluorescence (TONG; GIFFARD, 2012).

The fluorescence decay dynamics evaluated in HRM allows for more accurate discrimination between PCR products than simple comparison of melting temperatures (Tm). Tm values are defined by the size of the DNA sequences and the GC percentage (% GC). The correlation between Tm and % GC is due to the stronger bond between the strands of double-stranded DNA provided by the presence of three hydrogen bonds between GC pairs, compared to the two bonds between AT pairs (TONG; GIFFARD, 2012). In HRM, these characteristics are added to nucleotide substitutions in the definition of dissociation curves. Thus, it is possible to identify genetic polymorphisms and epigenetic alterations in DNA.

The document Naze et al. (2009) deals with multiplex PCR detection of DENV and CHIKV together, and WNV, followed by typing of DENV serotypes using the High Resolution Melting (HRM) strategy. Like the present invention, the method disclosed in Naze et al. (2009) deals with the development of assays to determine DENV serotypes. However, unlike the present invention, Naze et al. (2009) does not refer to the lineages within each serotype. It should be noted that Naze et al. (2009) only describes the differentiation of serotypes 1 to 4, without addressing the distinction of lineages and using reverse transcriptase in RT-PCR assays with TaqMan probes, a fact that distinguishes Naze et al. (2009) from the present invention.

In addition, it should be noted that the dissociation temperatures and primers used in the present invention and in Naze et al. (2009) are different, a critical difference between the two inventions. Before differentiating by HRM, the technique presented by Naze et al. requires a previous step, in which Naze et al. use a TaqMan probe for cDNA production. This step prior to HRM is not necessary in the present invention, since cDNA and HRM are performed in a single step, without the need for sample manipulation and using DNA intercalator. Thus, it can be said that the present invention has the following advantages over Naze et al.: (1) It is faster, since the entire serotype differentiation reaction occurs in a single PCR run; (2) It is more economical, since it uses DNA intercalator instead of the TaqMan probe, which is very expensive; (3) It has a reduced risk of contamination, since the technique is performed in a single step and requires less sample handling; (4) The dissociation temperatures for differentiation of serotypes 1 and 4 are very close in the protocol of Naze et al. (82.8° C. and 83° C.), and this proximity of temperature creates the need for a third stage of analysis, since the technique listed by Naze et al. requires the user to have in-depth knowledge of HRM analysis software, which is not required in the protocol of the present invention, given that the dissociation temperatures in the present invention are spaced far enough apart to be clearly distinct, which eliminates a step in the process, making it even easier and faster compared to Naze et al.

The document Villinger et al. (2016) discloses a method based on multiplex analysis for the detection of six genera in mosquitoes. The methodology applied involves PCR (Polymerase Chain Reaction) analysis followed by the High Resolution Melting (HRM) technique for the identification of these possible genera, which encompasses a range of specific viruses. It is therefore a generic reaction, making it impossible to differentiate between species within each genus, as well as between lineages. It should be noted that the present invention relates to a diagnostic method for humans, capable of detecting DENV serotypes and differentiating DENV-2 lineages I and II in a single reaction, while document D2 relates to a diagnostic method for mosquitoes.

Document CN110760614 discloses a diagnostic method for rapid detection and typing of Dengue viruses 1, 2, 3, and 4. Like the present invention, the aforementioned method begins its methodology with the design of degenerate primer oligonucleotides from a conserved region of the E protein among the four serotypes. These primers are used in the application of the real-time RT-PCR technique followed by high-resolution melting (HRM) curve analysis. The present invention, however, does not use degenerate primers, but rather primers in their conventional structure. Document CN110760614 proposes the detection of different serotypes, but does not address the distinction between lineages derived from DENV-2, as proposed in the present invention. In addition, the document still uses the reverse transcriptase enzyme in its assays, which differs from what is proposed in the present invention for the differentiation of DENV-2 lineages.

Document WO2010016071 discloses and describes as an invention the process of identifying “genomic signatures” for the differentiation of variants in very similar sequences of microorganisms. Conceptually, the term “genomic signature” refers to the characteristic frequency of nucleotides in a genome or sequence. Document WO2010016071 describes the process of identifying these signatures as potential tools for species differentiation, using the differentiation of HBV and HIV genotypes as examples. The document also treats the term genotype as synonymous with: variant, subtype, strain, and. However, when comparing WO2010016071 with the present invention, it should be noted that WO2010016071 does not describe a diagnostic method capable of detecting and differentiating lineages within the same serotype and genotype, a situation that considerably distinguishes the present invention from WO2010016071. As described, based on the identification of the genomic signature, it is possible to differentiate between microorganisms that are very similar in terms of genome analysis. In order for it to be read as a “diagnostic method”, the identification of these genomic signatures must be associated with visualization methods and not genome analysis. Thus, the genomic signature identification tool, as described in WO2010016071, based on computational and statistical analyses, can be used for diagnostic purposes when associated with other methodologies. However, each methodology requires a real test and standardization to assess its feasibility of use. For these reasons, the present invention differs considerably from WO2010016071. Another point to be highlighted is that WO2010016071 suggests targets that are different from those suggested in the present invention, since WO2010016071 uses HIV and HBV as models and the present invention uses dengue viruses and their different lineages.

As can be seen, no prior art document describes or suggests a method for distinguishing between the two lineages I and II of DENV-2 using real-time PCR followed by HRM.

The distinction between the two lineages I and II of DENV-2 and analysis of the high-resolution dissociation curve after thermal cycling has advantages over sequencing, a commercially used technique, among which is the fact that the process is faster, highly specific, sensitive, and lower in cost than sequencing.

In addition, this technique reduces sample handling and the chance of laboratory contamination, since the tests can be performed in a single step without opening the reaction tube, reducing reagent costs and favoring cases where the sample quantity is limited or difficult to access.

Another advantage of this technique is the innovation of the DENV-2 RNA genome amplification reaction without the use of reverse transcriptase. This reduces the cost and response time of each reaction.

The present invention may or may not use the reverse transcriptase enzyme for the identification of DENV-2 lineages, as data show that the Tag polymerase used in the process also has reverse transcriptase activity. It is therefore possible to add or not add the reverse transcriptase en. It is important to note that the inventive process is within the parameters of thermocycling developed, evaluated, and validated for the purpose of exploiting the reverse transcriptase activity of the DNA polymerase itself, thereby reducing the cost and time of the reaction.

The present invention allows for rapid, simple, and low-cost monitoring of the emergence and reemergence of DENV serotypes circulating in Brazil, as well as DENV-2 lineages with the pathogenic potential to cause more severe epidemics. The present invention provides a useful tool for medical follow-up of patients, since once dengue 2 lineage 2 is detected, the probability of hospitalization is much higher. Combined with this fact, the test results become important epidemiological monitoring tools for tracking the evolution of diseases in national and transnational territories. Currently, such differentiation is only possible through genetic sequencing, a technique that is very expensive, requires specialized personnel training, and takes much more time. With the technology described, the entire process takes 2 hours and costs much less than genetic sequencing. These facts reiterate the importance of the present invention.

### SUMMARY OF THE INVENTION

In one aspect, the present invention relates to a method for determining the presence or absence of the four serotypes of the dengue virus and differentiating between lineages I and II of serotype 2 of the dengue virus in a biological sample comprising the following steps: (a) amplifying nucleotide sequences defined among the sequences SEQ ID NO: 5-10, obtained or derived from a biological sample by real-time PCR using the primers as defined in sequences SEQ ID NO: 1, 2, 3, and 4, which when amplified generate at least one amplicon from a region of at least one nucleotide sequence as defined in sequences SEQ ID NO: 5-10 and wherein SEQ ID NO: 9 and 10 translate at least one of the amino acid sequences defined in SEQ ID NO: 11 and 12 and (b) identifying the amplicon produced according to the melting temperature (Tm) from high-resolution melting (HRM) curves specific for SEQ ID NO: 5, SEQ ID NO: 6, SEQ ID NO: 7, and SEQ ID NO: 8, SEQ ID NO: 9 and SEQ ID NO: 10. In one embodiment, there are specific substitutions in the amplicons produced, represented by two SNPs located in SEQ ID NO: 9 and 10, which can be translated into one of the amino acid sequences defined in SEQ ID NO: 11 and 12, and in which the first substitution is identified at position E129 (V-I) and the second substitution is at position E131 (L-Q), wherein SEQ ID NO: 11 has a valine amino acid at position E129 and leucine at position E131, and SEQ ID NO: 12 has an isoleucine amino acid at position E129 and glutamine at position E131. In a further embodiment, the Tms of SEQ ID NOs: 5 to 10 are, respectively, 79.25° C., 82.25° C., 81.19° C., 80.0° C., 79.9° C., and 78.6° C. In another additional embodiment, the method is performed in a single reaction step. In another additional embodiment, the method uses a DNA intercalator to generate the cDNA and determine the dissociation temperatures. In another additional embodiment, the biological sample may be urine, blood, saliva, a fragment of viscera, or any human or animal tissue.

In another aspect, the invention relates to the use of two pairs of primers, as defined in SEQ ID NO: 1, 2, 3, and 4, to determine the presence or absence of DENV serotypes, as defined in SEQ ID NO: 5, 6, 7, and 8, and to differentiate the serotype 2 strains of the dengue virus, defined in SEQ ID NO: 9 and 10, in a biological sample in which, additionally, the sequences SEQ ID NO: 9 and 10 can be translated into one of the amino acid sequences defined in SEQ ID NO: 11 and 12. In one embodiment, the primer pairs are defined by SEQ ID NO 1: 5′-CAT TGT GAC CTG TGC TAT GTT-3′ or SEQ ID NO 2: 5′-TTA CCT ACA GCG TGC TCT TCT-3′ or SEQ ID NO 3: 5′-CTT TCA ATA TGC TGA AAC GCG3′ or SEQ ID NO 4: 5′-TTC AAT ATC CCT GCT GTT GG-which, when amplified, generate an amplicon from a region of a nucleotide sequence of the 4 serotypes of DENV or lineages I and II of dengue serotype 2. In another aspect, the invention relates to the use of SNPs to distinguish lineages I and II of dengue serotype 2, defined in SEQ ID NO: 9 and 10. In one embodiment, the SNPs used to differentiate DENV-2 lineages, as defined in SEQ ID NO: 9 and 10, produce changes in the amino acid sequences located in the DENV-2 viral envelope, the first identified at position E129 (V-I) and the second at position E131 (L-Q).

In another aspect, the invention relates to a kit for detecting the four serotypes of the dengue virus and distinguishing two DENV-2 lineages, comprising (1) primers as defined in sequences SEQ ID NO: 1, 2, 3, and 4, which when amplified generate an amplicon from a region of a nucleotide sequence of each dengue serotype or of lineages I and II of dengue serotype 2; (2) amplification and HRM reagents.

### BRIEF DESCRIPTION OF THE FIGURES

The objective of the invention, together with additional advantages thereof, can be better understood by reference to the accompanying figures and the following descriptions:

FIG. 1 presents a graphical representation of the in silico analysis of the primers for detection and differentiation of lineages I and II of the Asian/American genotype of DENV-2, in terms of structure and positional entropy. (A) Forward Primer—(B) Reverse Primer. Areas in red indicate low positional entropy and consequently little flexibility in terms of structure, providing a more reliable prediction. Areas closer to violet reflect regions of higher positional entropy, greater flexibility in terms of structure, indicating a greater possibility of alternative structures forming. Source: RNAfold software-Gruber A R et al., 2008.

FIG. 2 shows the determination of dissociation temperatures for DENV-2 lineage I (79.9° C.) and lineage II (78.6° C.).

FIG. 3 describes (A) PCR reaction using GoScript RT (Promega, Madison, USA) with the addition of a pre-amplification reverse transcription step at 45° C. for 15 minutes. (B) PCR reaction without the use of external RT and without a prior reverse transcription step. Using the RT activity of HotStarTaq polymerase PLUS. Example of the detection capacity of known positive samples and, consequently, proving the reverse transcriptase activity of DNA polymerase.

FIG. 4 describes the graphical representation of the application of the newly standardized protocol to strains from the 1990s and after their emergence in 2007. (A) Dissociation temperature analysis, grouping samples into distinct peaks according to the temperature identified by the equipment. (B) Normalized decay curves showing three clusters-lineage I, lineage II, and lineage I associated with lineage II. (C) Another view of the normalization of the curves with a central emphasis on the control tube with both lineages.

FIG. 5 describes the graphical representation of the application of the newly standardized protocol against the 4 serotypes of the dengue virus. Distinct dissociation temperatures are observed, grouping the samples into 4 distinct peaks in duplicate, according to the temperature identified by the equipment.

FIG. 6A shows SEQ ID NO: 5 to 8, referring to DEN-1 to DENV-4, respectively. The parts in bold correspond to the beginning and end of each sequence. Each number corresponds to a distinct sequence. The colored parts are the modifications between the amplicon sequences. FIG. 6B represents SEQ ID NO: 9 and 10.

FIG. 7 shows SEQ ID NO: 11 and 12, referring to DENV-2 lineages I and II, respectively.

### DETAILED DESCRIPTION OF THE INVENTION

Although the present invention may be susceptible to different embodiments, a preferred embodiment is shown in the drawings and in the following detailed discussion, with the understanding that the present description should be considered an exemplification of the principles of the invention and is not intended to limit the present invention to what has been illustrated and described herein.

### Definitions

The term “biological sample” refers to fluids originating from the body. Body fluid samples are selected from urine, blood, saliva, for example. Samples of human visceral fragments from deceased individuals may also be used.

The term “nucleic acid”, “nucleotide sequence” or terms derived from these according to the present invention refers to any type of nucleic acid, such as DNA and RNA, and variants thereof, as well as combinations thereof, modifications thereof, including modified nucleotides, etc. The terms “nucleic acid” and “oligonucleotide” and “polynucleotide” are used interchangeably in the context of the present invention. Nucleic acids may be purified from natural sources, produced using recombinant expression systems, and optionally purified, chemically synthesized, etc. Where appropriate, for example in the case of chemically synthesized molecules, nucleic acids may comprise nucleoside analogs such as analogs having chemically modified bases or sugars, backbone modifications, etc. A nucleic acid sequence is represented in the 5′-3′ direction unless otherwise indicated. As used herein, the symbols for nucleotides and polynucleotides are those recommended by the IUPAC-IUB Commission on Biochemical Nomenclature (Biochem. 9:4022, 1970).

The term “Polymerase Chain Reaction” or PCR refers to a method in which a nucleic acid fragment is amplified. Primer oligonucleotides or primers are designed that have sequences complementary to the sequence to be amplified. PCR can be used to amplify RNA, DNA, or cDNA sequences.

The term “Amplicon” refers to a piece of DNA or RNA that is a product of amplification or replication events. It can be formed artificially, using various methods, including polymerase chain reactions (PCR) or ligase chain reactions (LCR), or naturally through gene duplication. As it refers to the product of an amplification reaction, amplicon is used interchangeably with the term “PCR product”.

The term “melting temperature” (Tm) refers to the melting temperature (Tm), or pairing temperature, as the temperature at which half of the DNA molecules are paired and the other half are not.

The term “HRM,” from “High Resolution Melting” refers to a post-PCR analysis method for identifying variations in nucleic acid sequences. The method is based on the detection of small differences in the melting curves generated by polymorphisms in the genome.

The term “SNP” refers to “single nucleotide polymorphism” and is used to designate natural variations that occur in the genome and involve, as the name implies, a single nucleotide. They may be associated with certain characteristics, thus functioning as molecular markers.

The term “intercalating” refers to substances capable of binding to double strands of DNA. Among the possible intercalating agents used, we can mention SYBR Green and EvaGreen.

Method for determining the presence or absence of the four serotypes of the dengue virus and differentiating between lineages I and II of serotype 2 of the dengue virus in a biological sample.

In one aspect, the present invention relates to a method for determining the presence or absence of the four serotypes of dengue virus and differentiating between lineages I and II of dengue virus serotype 2 in a biological sample, comprising the following steps: (a) amplifying nucleotide sequences defined among sequences SEQ ID NO: 5-10, obtained or derived from a biological sample by real-time PCR using the primers as defined in sequences SEQ ID NO: 1, 2, 3, and 4, which when amplified generate at least one amplicon of a region of at least one nucleotide sequence as defined in sequences SEQ ID NO: 5-10 and wherein SEQ ID NO: 9 and 10 translate at least one of the amino acid sequences defined in SEQ ID NO: 11 and 12, and (b) identifying the amplicon produced according to the melting temperature (Tm) from high-resolution melting (HRM) curves specific for SEQ ID NO: 5, SEQ ID NO: 6, SEQ ID NO: 7, and SEQ ID NO: 8, SEQ ID NO: 9, and SEQ ID NO: 10. In one embodiment, there are specific substitutions in the amplicons produced, represented by two SNPs located in SEQ ID NO: 9 and 10, which can be translated into one of the amino acid sequences defined in SEQ ID NO: 11 and 12, and wherein the first substitution is identified at position E129 (V→I) and the second substitution is at position E131 (L→Q). In an additional embodiment, the Tms of SEQ ID NO: 5 to 10 are, respectively, 79.25° C., 82.25° C., 81.19° C., 80.0° C., 79.9° C., and 78.6° C. In another additional embodiment, the method is performed in a single reaction step. In another additional embodiment, the method uses a DNA intercalator to generate the CDNA and determine the dissociation temperatures. In another additional embodiment, the biological sample may be urine, blood, saliva, a fragment of viscera, or any human or animal tissue.

The primers used in real-time PCR are as defined below:

SEQ ID NO: 1 (D2HRMF):
5′-CAT TGT GAC CTG TGC TAT GTT-3′
SEQ ID NO: 2 (D2HRMR):
5′-TTA CCT ACA GCG TGC TCT TCT-3′
SEQ ID NO: 3 (HRM_DENV_F):
5′-CTT TCA ATA TGC TGA AAC GCG-3′
SEQ ID NO: 4 (HRM_DENV_R):
5′-TTC AAT ATC CCT GCT GTT GG-3′.

The primers as defined in SEQ ID NO: 1 and 2 are used to differentiate DENV-2 lineages I and II, and the primers as defined in SEQ ID NO: 3 and 4 are used to differentiate the 4 dengue serotypes.

The amplicons of SEQ ID NO: 5 to 8 are as defined below:

| SEQ ID NO: 5: | |
|---|---|
| gacgggaaaa ccgtctatca atatgctgaa acgcgtgaga aaccgtgtgt caactggatc | 60 |
| acagttggcg aagagattct caagaggatt gctgaacggc caaggaccaa tgaaattggt | 120 |
| Tatggcgttc tcagatttct agccatccca ccaacagcag atagctttcc gagtcttggc | 180 |
| SEQ ID NO: 6: | |
| Ggtggttaga atatgctgaa acgcgagaga aaccgcgtat ccacctttca caacccctca | 60 |
| agggttggtg caaccggact tttctccggg aaaggaccct aagagattct tacggatggt | 120 |
| gcttgcattc tgcgagttct ttccatcccg ccaacagcag atcacatttt ggattctgaa | 180 |
| SEQ ID NO: 7: | |
| gacggctcga ccgtctttca atatgctgaa acgcgcgaga aaccgcgtgt caactgtctc | 60 |
| acagttggcg aagagattct caaaaggatt gctctcaggc cagggaccca tgaaactggt | 120 |
| gatggctttc atagcattcc taagatttct agccataccc ccaacagcag gaattttggc | 180 |
| SEQ ID NO: 8: | |
| ggcgaaaaac acgcctttca atatgctgaa acgcgagaga aaccgcgtgt cgactgtgca | 60 |
| acagctgaca aagagattct cacttggaat gctgcaggga cgaggaccat taaaactgtt | 120 |
| catggccctg gtggcgttcc ttcgtttcct aacaatccca ccaacagcag ggatattgaa | 180 |

SEQ ID NOS: 5, 6, 7, and 8 refer to sequences of DENV1, DENV-2, DENV-3, and DENV-4, respectively.

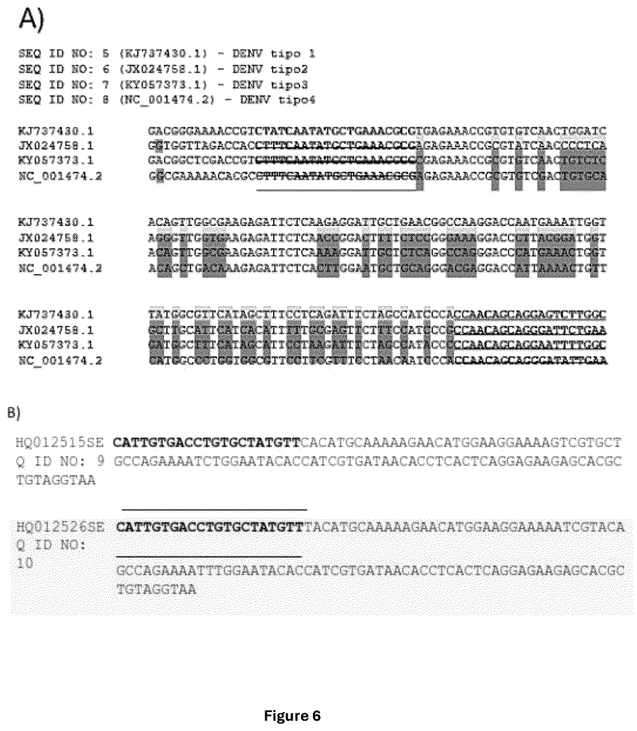

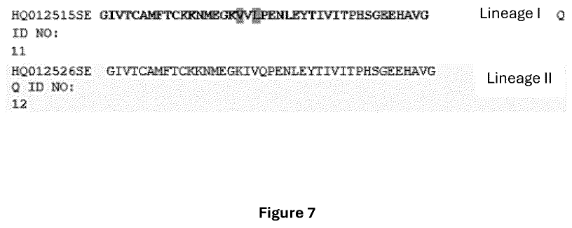

The representation of sequences SEQ ID NO: 9-12, which allow differentiation of lineages I and II of dengue virus serotype 2, are defined by the nucleotide sequences of SEQ ID NO: 9 and 10, and by the amino acid sequences of SEQ ID NO: 11 and 12 (FIG. 6B and FIG. 7).

Sequences SEQ ID NO: 9 to 12 are defined as follows:

| SEQ ID NO: 9: | |
|---|---|
| cattgtgacc tgtgctatgt tcacatgcaa aaagaacatg gaaggaaaag tcgtgctgcc | 60 |
| agaaaatctg gaatacacca tcgtgataac acctcactca ggagaagagc acgctgtagg | 120 |
| taa | 123 |
| SEQ ID NO: 10: | |
| cattgtgacc tgtgctatgt ttacatgcaa aaagaacatg gaaggaaaaa tcgtacagcc | 60 |
| agaaaatttg gaatacacca tcgtgataac acctcactca ggagaagagc acgctgtagg | 120 |
| taa | 123 |

SEQ ID NO: 11:
¹¹²Gly Ile Val Thr Cys Ala Met Phe Thr Cys Lys Lys Asn Met Glu Gly
Lys Val Val Leu Pro Glu Asn Leu Glu Tyr Thr Ile Val Ile Thr Pro
His Ser Gly Glu Glu His Ala Val Gly
SEQ ID NO: 12:
¹¹²Gly Ile Val Thr Cys Ala Met Phe Thr Cys Lys Lys Asn Met Glu Gly
Lys Ile Val Gln Pro Glu Asn Leu Glu Tyr Thr Ile Val Ile Thr Pro
His Ser Gly Glu Glu His Ala Val Gly

Sequences SEQ ID NO: 13 and 14 refer to the complete sequence of protein E of DENV 2 lineages I and II, respectively, where the differences between amino acids 129 and 131 are clear.

The melting temperatures (Tm) from high-resolution melting (HRM) curves specific to SEQ ID NO: 5, 6, 7, 8, 9, and 10 are, respectively, 79.25° C., 82.25° C., 81.19° C., 80.0° C., 79.9° C., and 78.6° C., respectively.

The sequences of each of the serotypes were aligned (FIG. 6) and the SNPs between the sequences were identified and marked in sequences SEQ ID NO: 5, 6, 7, and 8.

After performing the high-resolution (HRM) dissociation reaction, the differentiation of lineages occurs by identifying the substitutions of amino acid E129 (VI) of SEQ ID NO 13, in which valine is replaced by isoleucine, and in the amino acid at position E131 (L-Q), where leucine is replaced by glutamine, also in SEQ ID NO 13. The substitutions can be found in SEQ ID NO 14, which represents lineage II, while SEQ ID NO 13 represents lineage I. Thus, lineages I and II are differentiated by identifying the substituted amino acids.

It should be noted that, in addition to the changes listed, there are many other differences between lineages I and II.

### Use of Two Pairs of Primers

In another aspect, the invention relates to the use of two pairs of primers to determine the presence or absence of DENV serotypes, defined in SEQ ID NO: 5, 6, 7, and 8, and to differentiate between serotype 2 lineages of the dengue virus, defined in SEQ ID NO: 9 and 10 in a biological sample, wherein, additionally, the sequences SEQ ID NO: 9 and 10 can be translated into one of the amino acid sequences defined in SEQ ID NO: 11 and 12. In one embodiment, the primer pairs are defined by SEQ ID NO 1: 5′-CAT TGT GAC CTG TGC TAT GTT-3′ or SEQ ID NO 2: 5′-TTA CCT ACA GCG TGC TCT TCT-3′ or SEQ ID NO 3: 5′-CTT TCA ATA TGC TGA AAC GCG-3′ or SEQ ID NO 4: 5′-TTC AAT ATC CCT GCT GTT GG-which, when amplified, generate an amplicon from a region of a nucleotide sequence of the 4 DENV serotypes or lineages I and II of Dengue serotype 2.

### Use of SNPs

In another aspect, the invention relates to the use of SNPs to distinguish dengue serotype 2 lineages I and II, defined in SEQ ID NO: 9 and 10. In one embodiment, the SNPs used to differentiate DENV-2 lineages, as defined in SEQ ID NO: 9 and 10, produce changes in the amino acid sequences located in the DENV-2 viral envelope, the first identified at position E129 (V-I) and the second at position E131 (L-Q).

Confirmation of the changes resulting from the SNPs can also be performed using SEQ ID NO: 11 and 12 or SEQ ID NO: 13 and 14.

### Kit for Detecting the Four Serotypes of the Dengue Virus and for Distinguishing Two Lineages of DENV-2

In another aspect, the invention relates to a kit for detecting the four serotypes of dengue virus and for distinguishing two lineages of DENV-2, comprising (1) primers as defined in sequences SEQ ID NO: 1, 2, 3, and 4, which, when amplified, generate an amplicon from a region of a nucleotide sequence of each dengue serotype or of lineages I and II of dengue serotype 2; (2) amplification and HRM reagents.

The standard elements for a PCR reaction constitute the PCR kit itself, including: dNTPs, MgCl2, and enzyme (HotStarTaq Plus DNA Polymerase). In the case of the present invention, the non-use of reverse transcriptase enzyme for the differentiation of DENV-2 lineages I and II was evaluated, since the Taq Plus DNA Polymerase enzyme in the kit used also has reverse transcriptase action, under specific parameters used for temperature and cycling. In addition, the DNA intercalator, Q-solution, nuclease-free water, primers, and the target were used. The kit of choice for this assay is the Type-it HRM-PCR kit from Qiagen. For HRM, it is only necessary to use a PCR kit that is for single-tube PCR-HRM, configure the equipment for dissociation curve reading, and use primers that amplify a region containing SNPs.

### EXAMPLES

### Ethical Considerations

Samples isolated from patient serum stored in the sample bank of the Flavivirus Laboratory, IOC/FIOCRUZ, Regional Reference Center for arbovirus diagnosis in Brazil, were used. These samples are stored at −70° C. The study is within the objectives approved by the Research Ethics Committee (CAAE: 090249218.6.1001.5248) of the Oswaldo Cruz Foundation, Ministry of Health.

### DENV2 Strains

obtain the viral masses used in the standardization of the tests as positive controls, strains representative of the four DENV serotypes, as well as lineages I and II of the Southeast Asian genotype of DENV-2, were inoculated into Aedes albopictus clone C6/36 (ATCC® CRL1660™) cell culture according to Igarashi (1978). The cultures were inoculated with the strains separately and with mixtures of the two lineages for the DENV-2 lineage differentiation assays.

The strains characterized by Faria t al. (2013), BR59382/97 (Genbank accession number #HQ012515), representative of lineage I, was isolated from a male patient from the municipality of Natal, state of Rio Grande do Norte, who died in July 1997. The strain BR88034/07 (Genbank accession number #HQ012526), representative of lineage II, was isolated from a 12-year-old male patient from Nova Iguaçu, Rio de Janeiro, who presented with fever, prostration, headache, retro-orbital pain, myalgia, arthralgia and nausea.

In order to evaluate the PCR-HRM protocol's ability to differentiate between the two lineages present in the same tube and in the same test procedure, both lineages were inoculated into the same bottle.

### Standardization of the Assay

### Viral RNA Extraction

Strains representative of the four DENV serotypes, as well as DENV-2 lineages I and II, underwent viral RNA extraction using the commercial QIAMP Viral Mini Kit (QIAGEN, Inc., Valencia, USA), according to the manufacturer's recommendations. Briefly, 140 μl of sample were added to silica columns and mixed with 560 μl of AVL lysis buffer and 5.6 μl of carrier RNA, all provided by the kit. After washing with AW1 and AW2 buffers, the genetic material was eluted from the column by adding 60 μl of AVE buffer. The extracted viral RNA was stored at −70° C. until use.

### Determination and Evaluation of in Silico Primers

The design of the primers used for the detection of the four DENV serotypes (HRM_DENV_F and HRM_DENV_R, SEQ ID NO: 3 and 4, respectively) and differentiation of lineages I and II of the Asian/American genotype of DENV-2 (D2HRMF and D2HRMR, SEQ ID NO: 1 and 2, respectively) was performed using the online software Primer3. For this purpose, a dataset was generated from sequences available in the Virus Pathogen Resource (VIPR) and aligned using the software MEGAX (BETA) version 10.0 (http://www.megasoftware.net/). The primers were designed according to criteria that consider the number of nucleotides, amount of guanine (G) and cytosine (C), melting temperature (Tm), hybridization temperature, verification for the formation of hairpins and dimers, fragment size compatible with the HRM methodology, and presence of SNPs contained in SEQ ID NOS. 9 and 10, which can be translated into one of the amino acid sequences defined in SEQ ID Nos. 11 and 12, and in which the first substitution is identified at position E129 (V-I) and the second substitution is at position E131 (L-Q), in the amplification fragment that differentiates the two lineages of interest. The synthesis of the determined primers was performed by Integrated DNA Technologies—IDT.

### Evaluation of the Specificity of the Primers by PCR-HRM

The samples were subjected to real-time PCR methodology followed by high-resolution melting curve (HRM) analysis, using the commercial Type-it HRM PCR Kit (QIAGEN, Inc., Valencia, USA) in a 20 μl reaction containing: 10 μl of PCR HRM Master mix, 1.4 μl of forward and reverse primers at 0.7 μM, nuclease-free water, and RNA (1-50 ng, preferably 20 ng). The reactions were performed in a Rotor Gene Q thermocycler, using Gene ScreenClust HRM software for DNA intercalation.

### Establishment of Optimal Real-Time PCR Conditions and Specific Disociation Temperatures for Detection and Differentiation of the Two Lineages

The optimal conditions for real-time PCR associated with dissociation temperature analysis were established based on the protocol provided by the Type-it HRM PCR Kit (QIAGEN, Inc., Valencia, USA), where the amount and concentration of reagents, the amount of target material required for amplification, and a good HRM analysis were evaluated from the established viral masses in a two-hour reaction.

### Analysis of Optimal PCR Conditions Using the Reverse Transcriptase Activity of Hotstar Taq Polymerase Plus for Detection and Differentiation of the Two DENV-2 Lineages

In order to evaluate and utilize the reverse transcriptase activity of HotStarTaq polymerase PLUS, as described in the third edition of the handbook “Maximizing PCR and RT-PCR success-Addressing critical factors and new solutions” by QIAGEN (QIAGEN, Inc., Valencia, USA), representative samples isolated from lineages I and II were tested in a protocol without the use of reverse transcriptase and with the use of GoScriptRT Mix for 1 stepRTqPCR reverse transcriptase (Promega, Madison, USA) to verify the same results. For the protocol using RT GoScript (Promega), a pre-amplification reverse transcription step of 45° C. for 15 minutes was added to the protocol.

### Evaluation of Cross-Reactivity and Sensitivity of PCR-HRM

The cross-reactivity of PCR-HRM was evaluated against representative lineages of DENV-1, DENV-3, DENV-4, ZIKV, CHIKV, and vaccine FA in duplicate assays to rule out the possibility of overlap between dissociation temperatures.

For sensitivity analysis, PCR-HRM was tested with a panel (n=40) of strains isolated after the introduction of DENV-2 in the 1990s (n=20) and after its emergence in 2007 (n=20), previously characterized as belonging to lineages I and II of the Asian/American genotype of DENV-2, respectively (Table 1) and considered true positives. Sensitivity was obtained by the formula: a/a+b X 100%, where: a=number of true positives, b=number of false negatives.

Table 1: Representative strains of lineages I and II of the Asian/American genotype of DENV-2 isolated in the 1990s and after reemergence in 2007/2008 in Brazil.

| | Year of | | State of |
|---|---|---|---|
| Strain | isolation | Lineage | origin |
| BR39064/90 | 1990 | Lineage I | RJ |
| BR39122/90 | 1990 | Lineage I | RJ |
| BR39158/90 | 1990 | Lineage I | RJ |
| BR39279/90 | 1990 | Lineage I | RJ |
| BR41763/90 | 1990 | Lineage I | RJ |
| BR41764/90 | 1990 | Lineage I | RJ |
| BR41768/90 | 1990 | Lineage I | RJ |
| BR41769/90 | 1990 | Lineage I | RJ |
| BR41794/90 | 1990 | Lineage I | RJ |
| BR41802/90 | 1990 | Lineage I | RJ |
| BR41812/90 | 1990 | Lineage I | RJ |
| BR41821/90 | 1990 | Lineage I | RJ |
| BR41836/90 | 1990 | Lineage I | RJ |
| BR41837/90 | 1990 | Lineage I | RJ |
| BR41851/90 | 1990 | Lineage I | RJ |
| BR41856/90 | 1990 | Lineage I | RJ |
| BR41880/90 | 1990 | Lineage I | RJ |
| BR41898/90 | 1990 | Lineage I | RJ |
| BR41986/90 | 1990 | Lineage I | RJ |
| BR42003/90 | 1990 | Lineage I | RJ |
| BR42075/90 | 1990 | Lineage I | RJ |
| BR187/11 | 2011 | Lineage II | RJ |
| BR240/11 | 2011 | Lineage II | RJ |
| BR337/08 | 2008 | Lineage II | RJ |
| BR363/11 | 2011 | Lineage II | RJ |
| BR372/08 | 2008 | Lineage II | BA |
| BR486/11 | 2011 | Lineage II | RJ |
| BR772/10 | 2010 | Lineage II | RJ |
| BR780/10 | 2010 | Lineage II | RJ |
| BR785/10 | 2010 | Lineage II | RJ |
| BR934/10 | 2010 | Lineage II | RJ |
| BR1058/11 | 2011 | Lineage II | RJ |
| BR1067/11 | 2011 | Lineage II | RJ |
| BR1153/11 | 2011 | Lineage II | RJ |
| BR1433/11 | 2011 | Lineage II | RJ |
| BR2921/10 | 2010 | Lineage II | RJ |
| BR3778/10 | 2010 | Lineage II | RJ |
| BR7339/13 | 2013 | Lineage II | MS |
| BR7353/13 | 2013 | Lineage II | MS |
| BR7416/13 | 2013 | Lineage II | MS |
| BR7428/13 | 2013 | Lineage II | MS |

### Example 1

### In Silico Determination and Evaluation of Initiators for Detection of the 4 Serotypes of DENV and Differentiation of Lineages I and II of the Asian/American Genotype of DENV-2

The primers D2HRMF, D2HRMR, HRM_DENV_F, and HRM_DENV_R (Table 2) were analyzed by the RNAfold platform of the Institute of Theoretical Chemistry, University of Vienna, Austria, before being synthesized.

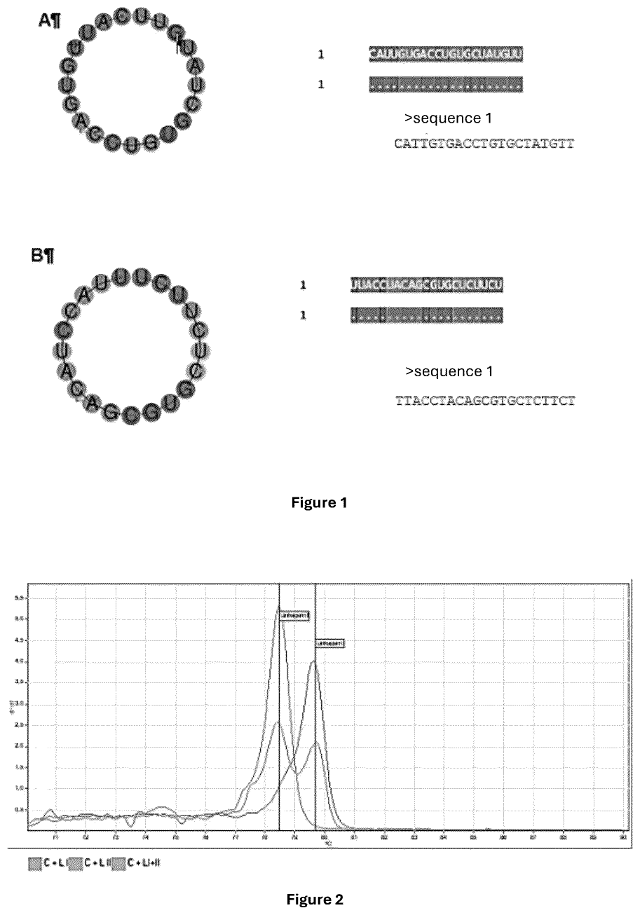

Analyses based on minimal free energy (MFE) and positional entropy showed a low probability of insufficient performance of the primers, according to the prediction of secondary structures. MFE analysis is related to the bases and sequence size, serving as an auxiliary tool capable of providing additional information before testing on equipment. The software performed the analysis based on programming algorithms described by Zuker and Stiegler (1981), using dynamic programming, without considering pseudoknots (FIG. 1).

Table 2—Sequence of primers for the PCR-HRM protocol for detection and differentiation of DENV-2 lineages I and II circulating in Brazil and differentiation of the four DENV serotypes.

| | | Tm (50 | |
|---|---|---|---|
| | | mM | |
| Primer | Sequence | NaCl) | Qt. |
| DRHRMF | 5′-CAT TGT GAC CTG | 53.1° C. | 21 bp |
| | TGC TAT GTT-3′ | | |
| D2HRMR | 5′-TTA CCT ACA GCG | 55.6° C. | 21 bp |
| | TGC TCT TCT-3′ | | |
| HRM_ | 5′- | 61.3° C. | 21 bp |
| DENV_F | CTTTCAATATGCTGAAACGCG-3′ | | |
| HRM_ | 5′-TTC AAT ATC CCT | 60.9° C. | 20 bp |
| DENV_R | GCT GTT GG-3′ | | |

### Evaluation of Primer Specificity by HRM PCR

The designed primers were synthesized for standardization tests and performed excellently. When evaluated in the Rotor Gene Q PCR-HRM equipment, the HRM_DENV_Fe and HRM_DENV_R primers proved capable of detecting and differentiating the four DENV serotypes from a 125 bp fragment. Similarly, the D2HRMF and D2HRMR primers, when evaluated on the Rotor Gene Q PCR-HRM equipment, were able to detect DENV-2 from the amplification of a 123-base pair fragment.

### Establishment of Optimal Real-Time PCR Conditions and Specific Dissociation Temperatures for the Detection and Differentiation of the Two Lineages

The standardization of optimal conditions for real-time PCR was initially performed on Applied Biosystems' ViiA7 equipment,—HRM Software Module for ViiA7 System, available from the Fiocruz Technology Platform Network, with a reaction time of one hour and forty minutes, and subsequently tested on Rotor Gene Q equipment, where it performed well in a two-hour reaction (Table 3).

Table 3—Thermocycling parameters established after standardization of the PCR-HRM protocol

| Step | Time | Temperature |
|---|---|---|
| 1 cycle | — | — |
| Initial activation | 5 minutes | 95° C. |
| 45 cycles | — | — |
| Denaturation | 10 seconds | 95° C. |
| Hybridization/Extension | 30 seconds | 55° C. |
| HRM | 0.1° C. filter | |

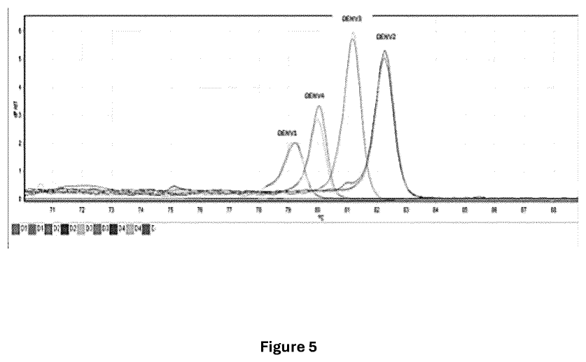

Based on the determination of the optimal conditions for reaction yield, it was possible to establish, in a specific manner, the different dissociation temperatures between serotypes and lineages and thereby determine the temperature difference between them. After analyzing the high-resolution dissociation curve in a single-tube post-PCR reaction lasting approximately two hours, the temperature was set at 79.9° C. for lineage I and 78.6° C. for lineage II (FIG. 2), resulting in a difference of 1.3° C. between lineages I and II of DENV-2. Regarding the four DENV serotypes, the temperature was set at 79.2° C. for DENV-1, 82.3° C. for DENV-2, 81.2° C. for DENV-3, and 80° C. for DENV-4 (FIG. 5).

### Analysis of Optimal PCR Conditions Using the Reverse Transcriptase Activity of HotStarTaq Polymerase PLUS for the Detection and Differentiation of the Two DENV-2 Lineages

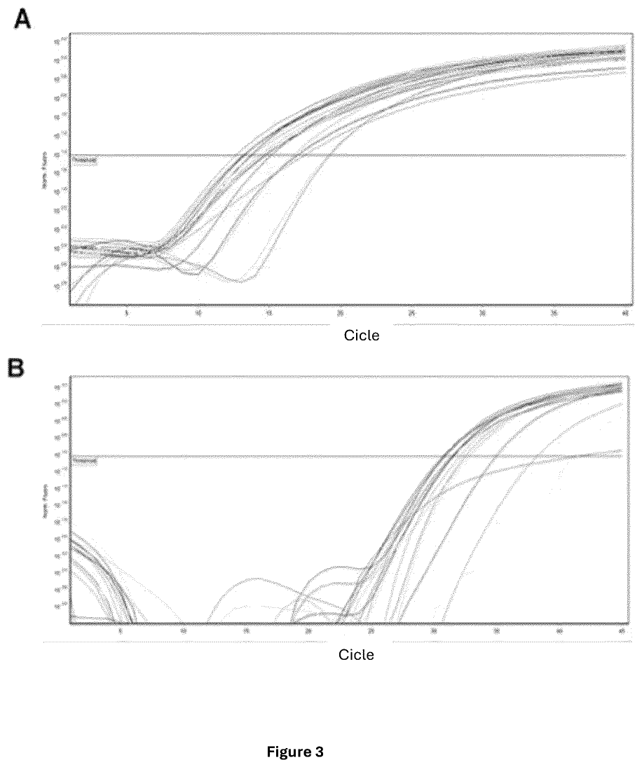

In order to use the reverse transcriptase activity present in HotStarTaq polymerase PLUS, we subjected the samples to PCR reactions with and without RT to assess whether the product would continue to be amplified and characterized as a positive sample. Thus, it was possible to observe that the protocol without the use of GoScript RT (Promega, Madison, using USA), only the reverse transcriptase activity of HotStarTaq polymerase PLUS, was able to detect the known positive samples (FIG. 3).

Evaluation of PCR-HRM Cross-Reactivity for the Differentiation of DENV-2 Lineages from Other Medically Important Arboviruses Circulating in the Country

The synthesized primers were included in specificity reactions to ensure that only the targets of interest were detected. HRM post-PCR reactions were performed with the same established parameters, using equipment with different reading filters for representative strains of ZIKV, DENV-1, DENV-3, DENV-4, CHIKV, and YEV vaccine. When analyzed on equipment with filters greater than 0.1° C., non-specific noises were observed. However, outside the range of dissociation temperatures sought (Table 5). In the equipment recommended by the Type-it HRM PCR Kit (QIAGEN, Inc., Valencia, USA), the Rotor gene Q, with a 0.1° C. filter, these noises were not observed, showing greater specificity for high-resolution readings. Thus, the primers proved to be specific for DENV-2 lineages I and II in a high-resolution analysis, showing no cross-reaction with the other arboviruses tested.

Evaluation of the Sensitivity of the Newly Standardized Method Against Strains Isolated after the Introduction of DENV-2 in the 1990s and after its Emergence in 2007

The newly standardized method was applied to strains isolated in the 1990s and after the emergence of DENV-2 in 2007 (n=40). In a two-hour reaction, it was able to detect and differentiate the lineages with specificity, based on different dissociation temperatures, grouping them into clusters in a graphical representation using Gene ScreenClust HRM software for DNA intercalating. In the reaction, the samples were tested in duplicate, with positive controls for each lineage, a negative control (no template control—NTC), and a control for the two lineages grouped in the same tube, in order to evaluate the simultaneous detection capacity.

Considering the truly positive samples (Table 2), PCR-HRM showed 100% sensitivity for both the detection of lineage I (20/20) and lineage II (20/20).

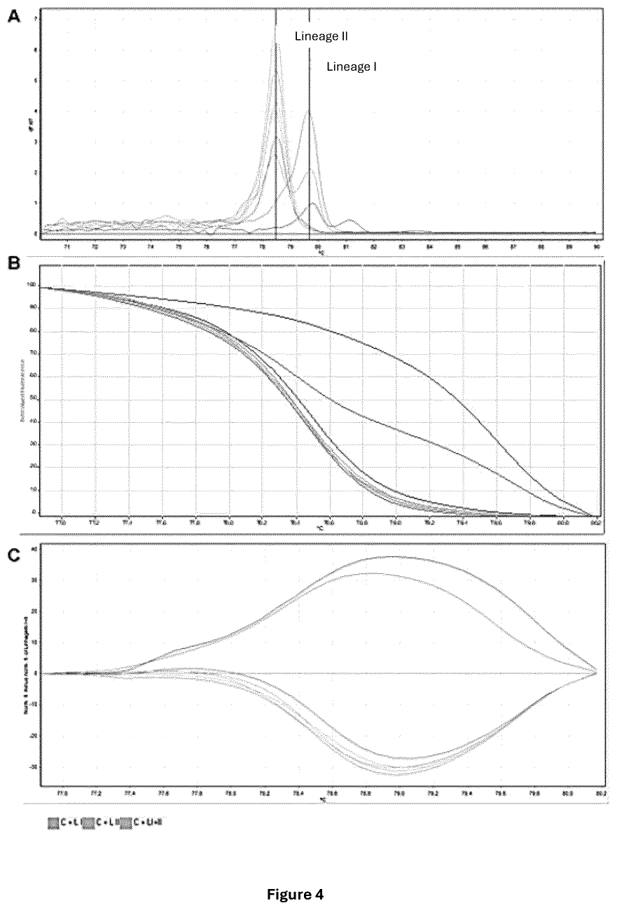

FIG. 4 (A) shows a representation of the amplification of the 20 representative samples of each lineage using the newly standardized protocol. All grouped correctly at the expected dissociation temperature, showing specific peaks for each lineage.

FIG. 4 (B) is a graphical representation of the amplification of each group of variants present in the assay, showing the fluorescence decay curve normalized according to the three controls determined for the equipment and grouping samples with the same dissociation temperature into distinct clusters.

FIG. 4 (C) demonstrates the possibility of another visualization of the clusters according to the normalization of the fluorescence decay curves, this time centering the control tube containing both lineages.

No co-infection by the two lineages was observed in the samples tested representing both periods.

### REFERENCES

Araújo, J M G.; Schatzmayr, H G. Aspectos Virais da Dengue (Viral Aspects of Dengue). In: Valle, D.; Pimenta,
D N.; Cunha, R V. (Org). Dengue Teorias e Práticas (Dengue Theories and Practices). Rio de Janeiro: FIOCRUZ, 2015; 171-185
Assenberg, R. et al. Crystal structure of a novel conformation state the Flavivirus NS3 protein: implications for polyprotein processing and viral replication. Journal of Virology. 2009; 83 (24): 12895-12906.
Banerjee, A; Paul, U K; Bandyopadhyay, Arup. Diagnosis of dengue fever: roles of different t laboratory test methods. Int J OfAdv In Med. 2018; 5 (2): 395-399.
Bharaj, P et al., Concurrent infections by all four dengue virus serotypes during an outbreak of dengue in 2006 in Delhi, India. Virology journal. 2008; 1-5.
BRISSON, MARNI et al. Optimization of Single and Multiplex Real Time PCR. In: A-Z of quantitative PCR. IUL Biotechnology Series. La Jolla, USA: Bustin, Stephen A, 2004.
Cam, B V et al., Prospective case-control study of encephalopathy in children with dengue hemorrhagic fever. Am J Trop Med. 2001; (65): 848-851.
Chambers T J, Hahn C S, Galler R, Rice C M et al. Flavivirus genome organization, expression and replication. Annu Rev Microbiol. 1990; (44): 649-688.
Chan, Hby; How, Ch. Definitive tests for dengue fever: when and which should I use? Singapore Med J. 2017; 58 (11): 632-635.
Chen R, Vasilakis N. Dengue-Quo tu et quo vadis? Viruses. 2011.; 3 (9): 1562-1608
Drumond B P, Mondini A, Schmidt D J, BronzoniRVdM, Bosch I, et al. 2013. Circulation of Different Lineages of Dengue Virus 2, Genotype American/Asian in Brazil: Dynamics and Molecular and Phylogenetic Characterization. PLOS ONE. 8 (3): 594-622.
Endy T P, et al., Epidemiology of inapparent and symptomatic acute Dengue virus infection: a prospective study of primary school children in Kamphaeng, Thailand. Am J Epidemiol. 2002; (156): 40-51.
Faria, NRC et al. Twenty years of DENV-2 activity in Brazil: molecular characterization and phylogeny of lineages isolated from 1990 to 2010. Plos Negl Trop Dis. 2013; 7 (3).
Fatima, Z et al. Serotype and genotype analysis of dengue virus by sequencing followed by phylogenetic analysis using samples from three mini outbreaks-2007-2009 in Pakistan. Bmc Microbiology. 2011; 11 (1): 1-8.
Igarashi, A. Isolation of a Singh's Aedes albopictus cell clone sensitive to Dengue and Chikungunya viruses. J Gen Virol. 1978. Sep 40 (3): 531-44.
Kuhn, R J. et al. Structure of Dengue Virus. Cell, Elsevier B V. 2002; 108, (5): 717-725.
Kuno, G, et al. Detecting artificial anti-dengue IgM immune complexes using an enzyme-linked immunosorbent assay. Am J Trop Med Hyg, 1987, 36 (1): 153-9.
Lanciotti R. et al., Molecular evolution and epidemiology of dengue-3 viruses. J Gen Virol. 1994; (1): 65-75
Lanciotti R S, Gubler D J, Trent D W. Molecular evolution and phylogeny of dengue-4 viruses. J Gen Virol. 1997; (9): 2279-84.
Lindenbach, B D.; Rice, C M. Flaviviridae: The viruses and their replication. In: Knipe D M, Howley, P M. Fields Virology, Philadelphia: Lippincott-Raven; 2001. 931-960.
Lupi, O, Carneiro, C G; Coelho, ICB. Manifestações mucocutâneas da dengue. Anais Brasileiros de Dermatologia (Mucocutaneous manifestations of dengue. Brazilian Annals of Dermatology), FapUNIFESP. 2007; 82 (4): 291-305
Naze, F; Roux K L, Schuffenecker I; Zeller H; Staikowsky F; Grivard P; Michault A; Laurent P. Simultaneous detection and quantification of Chikungunya, Dengue, and West Nile viruses by multiplex R T-PCR assays and Dengue virus typing using High Resolution Melting. Journal of Virological Methods. 2009. 1-7.
Nedjadi, T. et al. Tackling dengue fever: Current status and challenges. Virology Journal. 2015; Springer Nature.
Oliveira M F, Galvao Araujo J M, Ferreira O C, Ferreira D F, Lima D B, Santos F B, et al. 2010. Two lineages of dengue virus type 2, Brazil. Emerg Infect Dis., 16 (3), 576-8.
Rico-Hesse R. Origins of dengue type 2 viruses associated with increased pathogenicity in the Americas. Virology. 1997; (2): 244-255.
Rico-Hesse R. Molecular evolution and distribution of dengue viruses type 1 and 2 in nature. Virology, 1990; (174): 479-493.
Sampath A; Padmanabhan, R. Molecular targets for flavivirus drug discovery. Antiviral Research, Elsevier B V. 2009; (81): 6-15.
Staikowsky F, et al., Retrospective survey of chikungunya disease in Reunion Island hospital staff. Epidemiol. Infect. 2008; (136): 196206.
Selisko, et Barbara al. Regulation of Flavivirus RNA synthesis and replication. Current Opinion In Virology, Elsevier B V. 2014; (9): 74-83,
Sib Swiss Institute of Bioinformatics [homepage on the Internet]. Viral Zone: Flavivirus. [update 2016] Available from: https://viralzone. expasy. org/.
Singhi, S; Kissoon, N; Bansal, A. Dengue and dengue hemorrhagic fever: management issues in an intensive care unit. Jornal de Pediatria. 2007; 83 (7): 22-35.
Tewari, V V; Tewari, K; Mehta, R. Clinical and hematological profile of patients with dengue fever at a tertiary care hospital—an observational study. Mediterranean Journal Of Hematology And Infectious Diseases. 2018; 10 (1): 1-7.
TONG, S. Y. C.; GIFFARD, P. M. Microbiological Applications of High-Resolution Melting Analysis. Journal of Clinical Microbiology, v. 50, n. 11, p. 3418-3421 Nov. 2012.
Vasilakis N, Weaver S C. The history and evolution of human dengue emergence. Adv Virus Res. 2008; (72): 1-76
Villabona-Arenas C J, de Andrade-Zanotto P M. Evolutionary history of Dengue virus type 4: Insights into genotype phylodynamics. Infection, Genetics and Evolution, 2011; 11 (5), 878-885.
Villinger J; Ouso D; Mbaya M; Kipanga. Arbovirus and insect-specific virus discovery in Kenya by novel six genera multiplex high resolution melting analysis. Molecular Ecology Resources. 2017.
Weaver S C, Vasilakis N. Molecular evolution of dengue viruses: Contributions of phylogenetics to understanding the history and epidemiology of the preeminent arboviral disease. Inf Gen and Evol. 2009; (4): 523-541.
Zuker M &amp; Stiegler P. Optimal computer folding of large RNA sequences using thermodynamics and auxiliary information. Nucleic Acids Res. 1981. January 10; 9 (1): 133-148.

1. Method for determining the presence or absence of the four serotypes of the dengue virus and differentiating between lineages I and II of serotype 2 of the dengue virus in a biological sample comprising the following steps:
(a) amplifying nucleotide sequences defined among the sequences SEQ ID NO: 5-10, obtained or derived from a biological sample by real-time PCR using the primers as defined in the sequences SEQ ID NO: 1, 2, 3, and 4, which when amplified generate at least one amplicon from a region of at least one nucleotide sequence as defined in sequences SEQ ID NO: 5-10 and wherein SEQ ID NO: 9 and 10 translate at least one of the amino acid sequences defined in SEQ ID NO: 11 and 12;
(b) identify the amplicon produced according to the melting temperature (Tm) from high-resolution melting (HRM) curves specific to SEQ ID NO: 5, SEQ ID NO: 6, SEQ ID NO: 7 and SEQ ID NO: 8, SEQ ID NO: 9 and SEQ ID NO: 10.

2. Method according to claim 1, wherein the amplicons produced contain specific substitutions, represented by two SNPs located in SEQ ID NO: 9 and 10, which can be translated into one of the amino acid sequences defined in SEQ ID NO: 11 and 12, and wherein the first substitution is identified at position E129 (V→I) and the second substitution is at position E131 (L→Q), wherein SEQ ID NO: 11 has a valine amino acid at position E129 and leucine at position E131, and SEQ ID NO: 12 has an isoleucine amino acid at position E129 and glutamine at position E131.

3. Method according to claim 1, wherein the Tms of SEQ ID NO: 5 to 10 are, respectively, 79.25° C., 82.25° C., 81.19° C., 80.0° C., 79.9° C., and 78.6° C.

4. Method according to claim 1, wherein the aforementioned method is performed in a single reaction step.

5. Method according to claim 1, which uses a DNA intercalator to generate the cDNA and determine the dissociation temperatures.

6. Method according to any of claim 1, wherein the biological sample is urine, blood, saliva, visceral fragments, or any human or animal tissue.

7. Use of two pairs of primers, as defined in SEQ ID NO: 1, 2, 3, and 4, which is for determining the presence or absence of DENV serotypes, defined in SEQ ID NO: 5, 6, 7, and 8, and differentiating the lineages of serotype 2 of the dengue virus, defined in SEQ ID NO: 9 and 10 in a biological sample, wherein, additionally, the sequences SEQ ID NO: 9 and 10 can be translated into one of the amino acid sequences defined in SEQ ID NO: 11 and 12.

8. Use, according to claim 7, wherein the primer pairs are defined by the SEQ ID NO 1: 5′-CAT TGT GAC CTG TGC TAT GTT-3′ or SEQ ID NO 2: 5′-TTA CCT ACA GCG TGC TCT TCT-3′ or SEQ ID NO 3: 5′-CTT TCA ATA TGC TGA AAC GCG-3′ or SEQ ID NO 4: 5′-TTC AAT ATC CCT GCT GTT GG-which, when amplified, generate an amplicon from a region of a nucleotide sequence of the 4 DENV serotypes or lineages I and II of dengue serotype 2.

9. Use of SNPs which are used to distinguish strains I and II of dengue serotype 2, defined in SEQ ID NO: 9 and 10.

10. Use, according to claim 9, wherein the SNPs used to differentiate DENV-2 lineages, as defined in SEQ ID NO: 9 and 10, produce changes in the amino acid sequences located in the DENV2 viral envelope, the first identified at amino acid position E129 (V→I) and the second at amino acid position E131 (L→Q).

11. Kit for detecting the four serotypes of the dengue virus and for distinguishing two lineages of DENV-2, comprising:
primers as defined in sequences SEQ ID NO: 1, 2, 3, and 4, which, when amplified, generate an amplicon from a region of a nucleotide sequence of each dengue serotype or of lineages I and II of dengue serotype 2;
amplification and HRM reagents.


SEQUENCE LISTING (14 sequences)

SEQ ID NO: 1 - DNA, 21 bp, synthetic construct
  misc_feature 1..21: note = artificial
  source 1..21: mol_type = other DNA
cattgtgacctgtgctatgtt

SEQ ID NO: 2 - DNA, 21 bp, synthetic construct
  misc_feature 1..21: note = NA
  source 1..21: mol_type = other DNA
ttacctacagcgtgctcttct

SEQ ID NO: 3 - DNA, 21 bp, synthetic construct
  misc_feature 1..21: note = CTT TCA ATA TGC TGA AAC GCG
  source 1..21: mol_type = other DNA
ctttcaatatgctgaaacgcg

SEQ ID NO: 4 - DNA, 20 bp, synthetic construct
  misc_feature 1..20: note = NA
  source 1..20: mol_type = other DNA
ttcaatatccctgctgttgg

SEQ ID NO: 5 - DNA, 180 bp, Dengue virus
  source 1..180: mol_type = genomic DNA
gacgggaaaaccgtctatcaatatgctgaaacgcgtgagaaaccgtgtgtcaactggatcacagttggcgaagagattctcaagaggattgctgaacggccaaggaccaatgaaattggttatggcgttcatagctttcctcagatttctagccatcccaccaacagcaggagtcttggc

SEQ ID NO: 6 - DNA, 180 bp, Dengue virus
  source 1..180: mol_type = genomic DNA
ggtggttagaccacctttcaatatgctgaaacgcgagagaaaccgcgtatcaacccctcaagggttggtgaagagattctcaaccggacttttctccgggaaaggacccttacggatggtgcttgcattcatcacatttttgcgagttctttccatcccgccaacagcagggattctgaa

SEQ ID NO: 7 - DNA, 180 bp, Dengue virus
  source 1..180: mol_type = genomic DNA
gacggctcgaccgtctttcaatatgctgaaacgcgcgagaaaccgcgtgtcaactgtctcacagttggcgaagagattctcaaaaggattgctctcaggccagggacccatgaaactggtgatggctttcatagcattcctaagatttctagccatacccccaacagcaggaattttggc

SEQ ID NO: 8 - DNA, 180 bp, Dengue virus
  source 1..180: mol_type = genomic DNA
ggcgaaaaacacgcctttcaatatgctgaaacgcgagagaaaccgcgtgtcgactgtgcaacagctgacaaagagattctcacttggaatgctgcagggacgaggaccattaaaactgttcatggccctggtggcgttccttcgtttcctaacaatcccaccaacagcagggatattgaa

SEQ ID NO: 9 - DNA, 123 bp, Dengue virus
  source 1..123: mol_type = genomic DNA
cattgtgacctgtgctatgttcacatgcaaaaagaacatggaaggaaaagtcgtgctgccagaaaatctggaatacaccatcgtgataacacctcactcaggagaagagcacgctgtaggtaa

SEQ ID NO: 10 - DNA, 123 bp, Dengue virus
  source 1..123: mol_type = genomic DNA
cattgtgacctgtgctatgtttacatgcaaaaagaacatggaaggaaaaatcgtacagccagaaaatttggaatacaccatcgtgataacacctcactcaggagaagagcacgctgtaggtaa

SEQ ID NO: 11 - protein, 41 aa, Dengue virus
GIVTCAMFTCKKNMEGKVVLPENLEYTIVITPHSGEEHAVG

SEQ ID NO: 12 - protein, 41 aa, Dengue virus
GIVTCAMFTCKKNMEGKIVQPENLEYTIVITPHSGEEHAVG

SEQ ID NO: 13 - protein, 495 aa, dengue virus
MRCIGISNRDFVEGVSGGSTVDIVLEHGSCVTTMAKNKPTLDFELIKTEAKQPATLRKTCIEAKLTNTTTESRCPTQGEPSLNEEQDKRFICKHSMVDRGTGNGCGLFGKGGIVTCAMFTCKKNMEGKVVLPENLETTIVITPHSGEEHAVGNDTGKHGKEIKITPQSSITEAELTGTGTVTMECSPRTGLDFNEMVLLQMEEKATLVHRQTFLDLPLPTLPGADTQGSNTIQKETLVTFKNPHAKKQDVVVLGSQEGAMHTALTGATEIQMSSGNLLFTGHLKCRLRMDKLQLKGMSTSMCTGKFKIVKEIAETQHGTIVIRVQTEGDGSPCKIPFEIMDLEKRHVLGRLITVNPIVTEKDSPVNIEAEPPFGDSTIIIGVEPGQLKLNTFKKGSSIGQMFETTMRGAKRMAILGDTATDFGSLGGVFTSIGKALHQVFGAITGAAFSGVSTTMKILIGVIITTIGMNSRSTSLSVSLVLVGVVTLTLGAMVQA

SEQ ID NO: 14 - protein, 495 aa, dengue virus
MRCIGISNRDFVEGVSGGSTVDIVLEHGSCVTTMAKNKPTLDFELIKTEAKQPATLRKTCIEAKLTNTTTESRCPTQGEPSLNEEQDKRFICKHSMVDRGTGNGCGLFGKGGIVTCAMFTCKKNMEGKIVQPENLETTIVITPHSGEEHAVGNDTGKHGKEIKITPQSSTTEAELTGTGTVTMECSPRTGLDFNEMVLLQMEDKATLVHRQTFLDLPLPTLPGADTQGSNTIQKETLVTFKNPHAKKQDVVVLGSQEGAMHTALTGATEIQMSSGNLLFTGHLKCRLRMDKLQLKGMSTSMCTGKFKIVKEIAETQHGTIVIRVQTEGDGSPCKIPFEITDLEKRHVLGRLITVNPIVTEKDSPVNIEAEPPFGDSTIIVGVEPGQLKLNTFKKGSSIGQMFETTMRGAKRMAILGDTATDFGSLGGVFTSIGKALHQVFGAITGAAFSGVSTTMKILIGVIITTIGMNSRSTSLSVSLVLVGVVTLTLGAMVQA